(19) United States
(12) Patent Application Publication
(10) Pub. No.: US 2014/0235914 A1
(43) Pub. Date: Aug. 21, 2014

(54) Eggshell Catalyst Composites Containing Tungsten Oxide or Tungsten Oxide Hydrate

(71) Applicant: BASF CORPORATION, Florham Park, NJ (US)

(72) Inventor: Joseph C. Dellamorte, Beachwood, OH (US)

(73) Assignee: BASF Corporation, Florham Park, NJ (US)

(21) Appl. No.: 13/770,288

(22) Filed: Feb. 19, 2013

(51) Int. Cl. B01J 23/30; C07C 6/04

(52) U.S. Cl. CPC B01J 23/30; C07C 6/04
USPC 585/646; 502/254

(57) ABSTRACT

Provided are catalyst composites useful for the production of olefins during a metathesis reaction, as well as methods of making and using the same. Certain methods comprise a support comprising at least about 90% by weight silica; and an eggshell layer on the support comprising about 0.25 to about 10 wt. % tungsten in the form of tungsten oxide or tungsten oxide hydrate, based on the total weight of the catalyst composite.

### TECHNICAL FIELD

Aspects of the invention relate to catalyst composites, methods for preparing catalyst composites and methods of using the catalysts for reactions involving tungsten oxide, including mass transfer and/or selectivity limited reactions such as the metathesis of ethylene and 2-butene to form propylene.

### BACKGROUND

Olefin metathesis reactions generally involve the redistribution of alkenes by cleavage and regeneration of C—C double bonds to form different olefins. One example of such a reaction is the formation of propylene from ethylene and 2-butene. This type of reaction has been popular, due to the relatively low rate of undesired byproducts and/or wastes. As a result, several catalysts have been prepared to aid in metathesis reactions.

One catalyst that has been used is tungsten oxide, generally in the form of particles for fixed bed reactors. However, commercially available tungsten oxide catalyst particles show suppressed activity due to mass transfer limitations. That is, much of the catalytic potential is not utilized, because the catalyst is not more readily available during the chemical reaction. Thus, there is a need for catalyst composites which feature greater amounts of tungsten oxide available for reaction so that higher reaction rates may be achieved.

### SUMMARY

One aspect of the present invention pertains to a catalyst composite comprising a support comprising silica and a tungsten-based eggshell layer on the support. In one or more embodiments of this aspect, the catalyst composite comprises a support comprising at least about 90% by weight silica and an eggshell layer on the support comprising about 0.25 to about 10 wt. % tungsten in the form of tungsten oxide or tungsten oxide hydrate, based on the total weight of the catalyst composite.

In some embodiments, the tungsten oxide or tungsten oxide hydrate has a specific average crystal size, such as an average crystal size of less than or equal to about 1 micron or less than or equal to about 100 nm.

One or more embodiments provide that the catalyst composite has certain characteristics in its X-ray diffraction pattern. In some embodiments, the catalyst composite exhibits an X-ray diffraction pattern comprising a peak at a two-theta value of about 16±0.5 degrees and/or about 26±0.5 degrees. In some embodiments, the catalyst composite exhibits the X-ray diffraction pattern as shown in FIG. 1.

The amount of tungsten in the catalyst composite may vary. For example, in some embodiments, the catalyst composite comprises about 0.5 to about 7 wt. % tungsten in the form of tungsten oxide or tungsten oxide hydrate.

In one or more embodiments, the eggshell layer has an average depth of 20 to 500 micrometers or an average depth of 100 to 300 micrometers.

Another aspect of the present invention pertains to a method of making a catalyst composite. In various embodiments, the method comprises providing a support comprising at least about 90% silica, impregnating the support with water, and impregnating the support with a solution comprising ammonium paratungstate and hydrochloric acid to provide a catalyst composite comprising silica and tungsten oxide hydrate.

In one or more embodiments, the method further comprising drying the catalyst composite and calcining the catalyst composite at a temperature from about 150° C. to about 550° C. to provide a catalyst composite comprising silica and tungsten oxide. Alternatively, the catalyst composite may be placed in the reactor in uncalcined form and may be calcined by the conditions in the reactor.

In some embodiments, the catalyst composite comprises about 0.25 to about 10 wt. % tungsten oxide hydrate or about 0.5 to about 7 wt. % tungsten oxide hydrate prior to calcination. In some embodiments, the catalyst composite after calcination may comprise about 0.25 to about 10 wt. % tungsten oxide or about 0.5 to about 7 wt. % tungsten oxide.

One or more embodiments provide that the support is impregnated by spraying the water on the support and/or by spraying the solution comprising ammonium paratungstate and hydrochloric acid on the support.

Various relative amounts of water and ammonium paratungstate/hydrochloric acid solution may be used. In some embodiments, the support is impregnated with about ¼ to about 1 pore volume of water and/or about ¼ to about 1 pore volume of the solution comprising ammonium paratungstate and hydrochloric acid.

The amount of ammonium paratungstate in the hydrochloric acid solution may also be varied. In some embodiments, the molar ratio of the ammonium paratungstate to the hydrochloric acid in the solution is less than about 1:50.

Also provided is a catalyst composite obtained by any of the methods described herein. In some embodiments, the catalyst composite is obtained by impregnation with water and ammonium paratungstate/hydrochloric acid solution. The catalyst composite may comprise tungsten oxide hydrate, or may be calcined to comprise tungsten oxide.

Yet another aspect of the present invention relates to a method of reacting olefins in a metathesis reaction, the method comprising contacting a stream comprising olefins with any of the catalyst composites described herein. Exemplary metathesis reactions include, but are not limited to: the production of propylene from ethylene and 2-butene; the production of propylene from a mixture of ethylene, 2-butene and 1-butene; the production of propylene from ethylene and 2-pentene; the production of propylene from a mixture of ethylene, butenes and pentenes; and the production of 3-hexene and/or 1-hexene from 1-butene.

### BRIEF DESCRIPTION OF THE DRAWINGS

FIG. 1 is an X-ray diffraction pattern of an eggshell catalyst layer in accordance with one or more embodiments of the invention prior to calcination;

FIG. 2 is an X-ray diffraction pattern of an eggshell catalyst layer in accordance with one or more embodiments of the invention after calcination; and

FIG. 3 shows the propylene productivity of two catalysts formed in accordance with one or more embodiments of the invention and two comparative catalysts; and

FIG. 4 is a photograph of a catalyst composite formed according to one or more embodiments of the invention.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

### Catalyst Composite

One aspect of the invention relates to a catalyst composite generally based on tungsten oxide or tungsten oxide hydrate on an inert silica support. The catalyst composite comprises a support comprising at least about 90% by weight silica and an eggshell layer on the support comprising about 0.25 to about 10 wt. % tungsten in the form of tungsten oxide or tungsten oxide hydrate, based on the total weight of the catalyst composite. It has been discovered that such eggshell layers provide increased tungsten oxide/tungsten oxide hydrate availability, thus making catalysts employing such layers suitable for mass transfer and/or selectivity-limited reactions such as metathesis reactions. In one or more embodiments, higher reaction rates are achieved with other possible benefits in propylene productivity, catalyst life, and future reactor design flexibilities.

As used herein, the term “eggshell layer” or “eggshell catalyst layer” refers to a thin layer of catalytically active material on the outer regions of the support. It is not necessarily a layer that rests exclusively over the support, but rather that the outer regions of the support contain catalytically active material. In one or more embodiments, the eggshell layer is continuous around the support. In one or more embodiments, the catalyst penetrates the support at a depth of less than about 500, 450, or 400 μm. In some embodiments, the eggshell layer has an average depth of about 20 to 500, 75 to 450, or 100 to 300 micrometers.

As used herein, the term “tungsten oxide hydrate” is used synonymously with “tungstite,” and is represented by the formulas WO₃.H₂O, WO₃.2H₂O, and/or WO₃₀.⅓H₂O. In one or more embodiments, the tungsten oxide hydrate used in the catalyst composites is WO₃.H₂O.

Embodiments of the above aspect include variations in the average crystal size of the tungsten oxide or tungsten oxide hydrate. In some embodiments, the tungsten oxide or tungsten oxide hydrate has an average crystal size of less than or equal to about 1 micron, 900 nm, 800 nm, 700 nm, 600 nm, 500 nm, 400 nm, 300 nm, 200 nm, 100 nm, 90 nm, 80 nm, 70 nm or 60 nm.

In some embodiments, the loading of the catalyst is varied. Thus, in some embodiments, the catalyst composite comprises about 0.25 to about 10 wt. % tungsten in the form of tungsten oxide or tungsten oxide hydrate. The loading of tungsten oxide catalyst may have an upper limit of 10, 9, 8, 7, 6, 5, 4, 3, 2 or 1 wt. % and may have a lower limit of 5, 4, 3, 2, 1.5, 1, 0.75, 0.5 or 0.25 wt. %.

The amount of silica in the support may also be varied. The support may comprise at least 50, 60, 70, 80, 90, 95, 96, 97, 98, 99, 99.5 wt. % silica or more. In one or more embodiments, the support comprises at least 90% by weight silica. The support may also include additional oxide components such as alumina, titania, and/or zirconia, as well as alkali or alkaline earth metals such as sodium, potassium, calcium and/or magnesium.

In some embodiments, X-ray diffraction can be used to characterize the catalyst composite material. In one or more embodiments where the tungsten oxide is in the form of tungsten oxide hydrate, there may be a peak at a two-theta value of about 16±0.5 degrees and/or a peak at a two-theta value of about 26±0.5 degrees.

### Preparation

Another aspect of the invention relates to a process for making one or more of the catalyst composites with an eggshell catalyst layer described herein generally using oxide precipitation/impregnation.

The method comprises providing a support comprising at least about 90% silica, impregnating the support with water, and impregnating the support with a solution comprising ammonium paratungstate (APT) and hydrochloric acid (HCl) to provide a catalyst composite comprising silica and tungsten oxide hydrate.

Generally, if ammonium paratungstate is added to HCl, tungsten oxide hydrate will precipitate out of solution when the APT/HCl comes into contact with the water. When ammonium paratungstate is dissolved in hydrochloric acid and the subsequent solution is then added to a large excess of water, nanometer sized tungsten oxide hydrate crystals can be formed. In one or more embodiments, the support is impregnated with about ¼ to about 1 pore volume of water. In further embodiments, the support is impregnated with about ½ pore volume of water. In some embodiments, the support is impregnated with about ¼ to about 1 pore volume of the solution comprising ammonium paratungstate and hydrochloric acid. In further embodiments, the support is impregnated with about ½ pore volume of the APT/HCl solution.

In one or more embodiments, impregnation of the catalyst onto the support may be repeated until a desired catalyst loading is achieved. In some embodiments, each impregnation results in higher amounts of deposited catalyst by increasing the amount of APT in the HCl. Thus, in one or more embodiments, the amount of APT in the HCl is increased until the solubility limit of APT in HCl is reached. In some embodiments, the molar ratio of the ammonium paratungstate to the hydrochloric acid in the solution is less than about 1:50, i.e. the solution is less than 2 mol % ammonium paratungstate.

Some embodiments of the methods described herein may further comprise drying the catalyst composite and/or calcining the catalyst composite. In one or more embodiments, the catalyst composite is calcined at a temperature from about 150° C. to about 550° C. to provide a catalyst composite comprising silica and tungsten oxide. In one or more embodiments, the catalyst composite may be calcined for a period of 1 to 24 hours. In some embodiments, the catalyst composite is calcined for 1 to 6 hours or 2 to 4 hours.

While not wishing to be bound to any particular theory, it is thought that the tungsten oxide is in the hydrate form after impregnation. Calcining the catalyst composite results in non-hydrate tungsten oxide. Thus, in one or more embodiments, the final catalyst composite comprises about 0.25 to about 10 wt. % tungsten in the form of tungsten oxide. In further embodiments, the catalyst composite comprises about 0.5 to about 7 wt. % tungsten in the form of tungsten oxide hydrate.

Even if the catalyst composite is not calcined prior to use in the reactor, in some situations, use of the catalyst composite in certain reactions may calcine the catalyst composite. In such situations, the catalyst composite may initially have a tungsten oxide hydrate eggshell layer on the support, but the tungsten oxide hydrate may dehydrate to tungsten oxide as the catalyst composite is exposed to elevated reaction temperatures.

There are several variants in the processes described. In one or more embodiments, the support is impregnated by spraying the water on the support. Spraying water may result in very thin and very uniform eggshell coatings. In some embodiments, the solution comprising ammonium paratungstate and hydrochloric acid is impregnated by spraying the solution on the support. In one or more embodiments, the support is impregnated with about ½ pore volume of water and about ½ pore volume of the solution comprising ammonium paratungstate and hydrochloric acid. In some embodiments, the molar ratio of the ammonium paratungstate to the hydrochloric acid in the solution is less than about 1:50.

Yet another aspect of the invention relates to catalyst composites produced by any of the preparation methods described herein.

### Application

In one or more embodiments, the catalyst composites described herein may be used for metathesis reactions. Accordingly, one aspect of the invention relates to a method of reacting olefins in a metathesis reaction. The method comprises contacting a stream comprising the method comprising contacting a stream comprising olefins and a catalyst composite described herein. In one or more embodiments, the catalyst composite comprises a support comprising at least about 90% by weight silica and an eggshell layer on the support comprising about 0.25 to about 10 wt. % tungsten in the form of tungsten oxide or tungsten oxide hydrate, based on the total weight of the catalyst composite.

Another aspect of the invention relates to methods of metathesizing olefins. In some embodiments, the method comprises contacting a stream comprising olefins with the catalyst composite produced by providing a support comprising at least about 90% silica, impregnating the support with water, and impregnating the support with a solution comprising ammonium paratungstate and hydrochloric acid to provide a catalyst composite comprising silica and tungsten oxide hydrate. In one or more embodiments, the method comprises contacting a stream comprising olefins with the catalyst composite produced by providing a support comprising at least about 90% silica, impregnating the support with water, impregnating the support with of a solution comprising ammonium paratungstate and hydrochloric acid to provide a catalyst composite comprising silica and tungsten oxide hydrate, and drying the catalyst composite and calcining the catalyst composite at a temperature from about 150° C. to about 550° C. to provide a catalyst composite comprising silica and tungsten oxide

In some embodiments, the catalyst composites may be more broadly suitable for any reaction involving tungsten oxide, particularly mass transfer or selectivity-limited reactions.

In one or more embodiments, propylene is the desired product. In some embodiments, propylene may be produced from ethylene and 2-butene. In further embodiments, the reactor may be run with a mix of ethylene, 2-butene and 1-butene in the presence of a catalyst described herein and an isomerization catalyst. Isomerization catalysts include, but are not limited to, catalysts comprising magnesium oxide (MgO). In some other embodiments, propylene may be produced from ethylene and 2-pentene. In further embodiments, the reactor may be run with a mix of butenes, pentenes and ethylene in the presence of a catalyst described herein and an isomerization catalyst.

In yet other embodiments, 3-hexene may be produced. In some embodiments, 3-hexene is produced from 1-butene and the 3-hexene is isomerized to give 1-hexene.

### EXAMPLES

Without intending to limit the invention in any manner, embodiments of the present invention will be more fully described by the following examples.

### Example 1

A catalyst composite was prepared using ⅛″ extrudates composed of >90% silica. The extrudates were spray impregnated with 45% pore volume of de-ionized water while tumbling. The extrudates were allowed to tumble for 30 minutes. Ammonium paratungstate (APT) was added to hydrochloric acid (HCl) to form a 2 mol % APT in HCl solution. This mixture was stirred vigorously for 1 hour to ensure good dissolution of the APT into the HCl. The water-impregnated extrudates were then impregnated with enough APT/HCl solution to reach incipient wetness (i.e. to reach the full pore volume). The resulting catalyst was then dried in vacuum to a loss on drying of less than 2%. Depending on the nominal weight percentage required for the catalyst sample, this impregnation procedure was repeated. After the final impregnation and drying, the catalyst sample was characterized using X-ray diffraction (to determine crystal phases) and/or X-ray fluorescence (to determine WO₃ loading).

X-ray diffraction measurements were taken with a PANalytical MPD X'Pert Pro diffraction system. Cu_{Kα} radiation was used in the analysis with generator settings of 45 kV and 40 mA. The optical path consisted of a ¼° divergence slit, 0.04 radian soller slits, 15 mm mask, ½° anti-scatter slits, the sample, 0.04 radian soller slits, Ni filter, and an X'Celerator position sensitive detector. The X-ray diffraction samples were first prepared by grinding in a mortar and pestle and then backpacking the sample into a round mount. The data collection from the round mount covered a range from 10° to 70° 2θ using a step scan with a step size of 0.033° 2θ and a count time of 120 s per step.

X-ray fluorescence measurements were taken with a PANalytical PW2400. The samples were first calcined at 500° C. After cooling, 3.0 grams were ground with 2.0 grams of cellulose binder to ˜10 microns using a pulverizer. The sample-binder mixture was transferred to an aluminum cup and pressed at 30,000 psi to form a pellet, which was analyzed for W by an XRF spectrometer using the W La line with a LiF crystal.

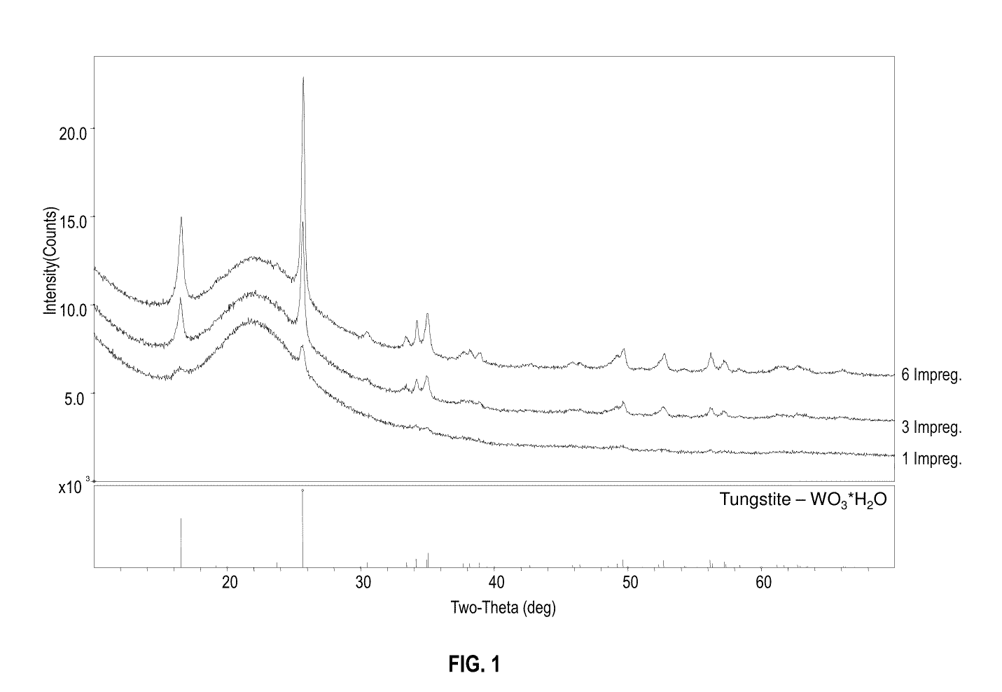

The X-ray diffraction patterns of the catalyst composites were obtained for the composite after one, three and six impregnations, and are shown in FIG. 1. As seen in the X-ray diffraction pattern, there is a peak at a two-theta value of about 16±0.5 degrees and a peak at a two-theta value of about 26±0.5 degrees. These peaks are thought to correspond to the presence of tungsten oxide hydrate. Accordingly, in one or more embodiments, the catalyst composite comprises tungsten oxide hydrate, and the catalyst composite may have a peak at a two-theta value of about 16±0.5 degrees and/or a peak at a two-theta value of about 26±0.5 degrees. In various embodiments, an X-ray diffraction pattern of the catalyst composite may also include additional peaks. The peaks may also have a variation of ±1, ±0.75±0.5, ±0.3, ±0.2 or ±0.1 degrees.

Furthermore, in some embodiments of the present invention, the catalyst composite exhibits the X-ray diffraction pattern as shown in FIG. 1. The phrase “exhibits the X-ray diffraction pattern as shown in FIG. 1” means that at least one of the peaks of a reference catalyst composite substantially overlaps with at least one peak as shown in FIG. 1. The at least one peak may be in the X-ray diffraction pattern shown for the 6-impregnation catalyst composite, the 3-impregnation catalyst composite or the 1-impregnation catalyst composite. Of course, some variation in the peak locations and intensity is possible depending on the X-ray diffraction technique. It is not necessary for a reference catalyst composite to have all of the peaks as shown in FIG. 1, nor is it necessary for a catalyst composite to only have those peaks shown in FIG. 1. However, in one or more embodiments, the catalyst composite of one or more aspects of the invention includes one, two, three, four, five or all of the peaks shown in FIG. 1. In some embodiments, the catalyst composite exhibits the X-ray diffraction pattern in FIG. 1 as shown for the 6-impregnation catalyst, the 3-impregnation catalyst or the 1-impregnation catalyst.

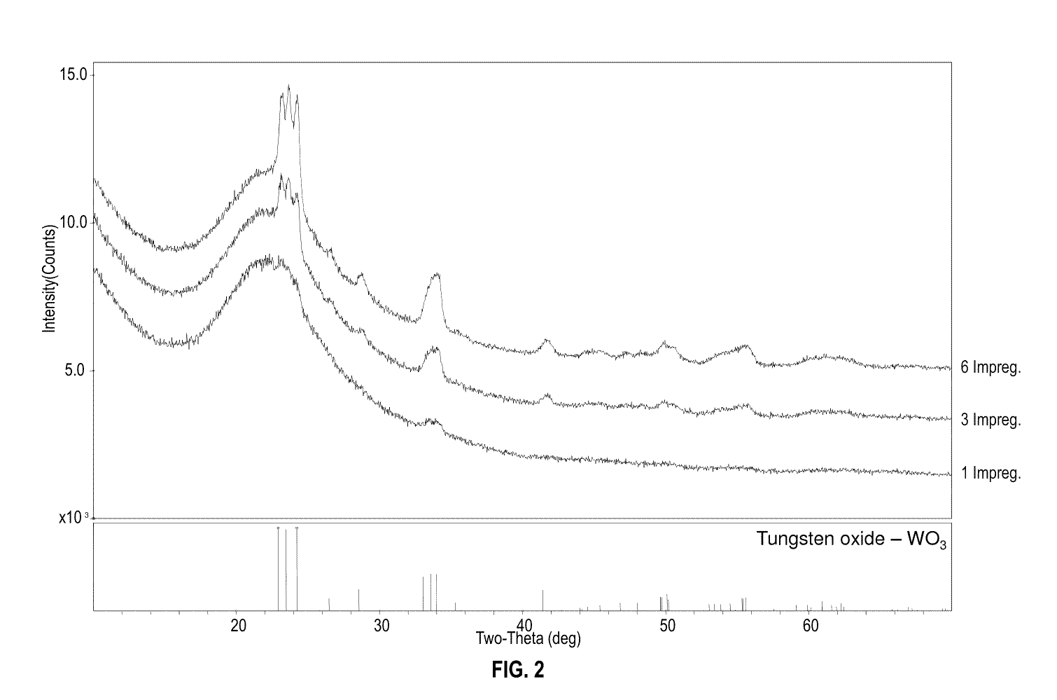

The catalyst composites prepared above were then each calcined at 500° C. for about 2 hours. The X-ray diffraction pattern of each catalyst composite was again obtained, and is shown in FIG. 2. As can be seen in the figure, the peaks have changed from the composites before calcination. It is thought that the peaks shown in FIG. 2 correspond to tungsten oxide. Table 1 below shows the amount of tungsten oxide after one, three and six impregnations, as measured by X-ray fluorescence.

TABLE 1
Amount of WO₃

| Composite No. | No. Impreg. | XRF - wt % WO₃ |
|---|---|---|
| 1A | 1 | 1.0% |
| 1B | 3 | 2.4% |
| 1C | 6 | 4.4% |

As can be seen from Table 1, the catalyst composite with one impregnation (composite 1A) contained 1.0% wt % WO₃, the composite with three impregnations (composite 1B) contained 2.4% wt % WO₃, and the composite with six impregnations (composite 1C) contained 4.4% wt % WO₃.

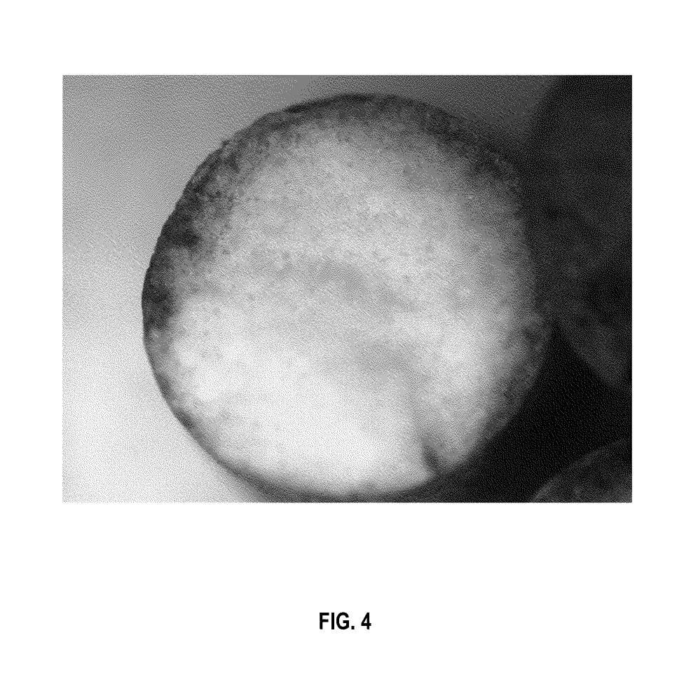

Catalyst composite 1A was then cross-sectioned, which is shown in FIG. 4. The darkened areas of the particle show where the eggshell catalyst layer is, and thus shows the presence of the tungsten oxide. The light areas show the areas of silica without tungsten oxide. As can be seen in FIG. 4, the catalyst composite exhibits the tungsten oxide on the outer regions of the particle, making it more readily available during a chemical reaction.

### Example C1

A catalyst composite was prepared based on WO 02/100535, which is incorporated by reference herein. The catalyst composite featured high purity silica granules impregnated with ammonium metatungstate. The resulting catalyst contained about 8.1 weight-% WO₃ and 0.1 weight-% potassium. Example C1 is considered comparative because the tungsten oxide catalyst is not contained as an eggshell catalyst layer.

### Example C2

A comparative catalyst composite was prepared using the same support as in Example 1, but the tungsten oxide was loaded using conventional methods, otherwise known as incipient wetness impregnation. A solution containing 0.06 mol % ammonium metatungstate in deionized water was mixed for 30 minutes using a magnetic stir bar. The solution was then sprayed onto silica extrudates to fill 90% of the pore volume while stirring. After complete addition of the solution, the catalyst composite was dried under vacuum and while tumbling at 120° C. to a loss on drying of <2%. The resulting catalyst composite contained 7.7 weight-% WO₃ Example C2 is considered comparative because the tungsten oxide catalyst is not contained as an eggshell catalyst layer.

### Example 2

The activities of the 1 wt. % and 4.4 wt. % WO₃ catalyst composites from Example 1 were measured, as well as the comparative catalyst composites from Examples C1 and C2. The conditions of the reaction are shown below in Table 2.

TABLE 2
Reaction Conditions

WHSV = 20 h⁻¹
300° C.; 400 psig
4 g Full size particles
Ethylene to 1-Butene feed ratio of 1.8
Mixed bed with 3 parts by weight of MgO and 1 part
by weight of the WO₃/silica

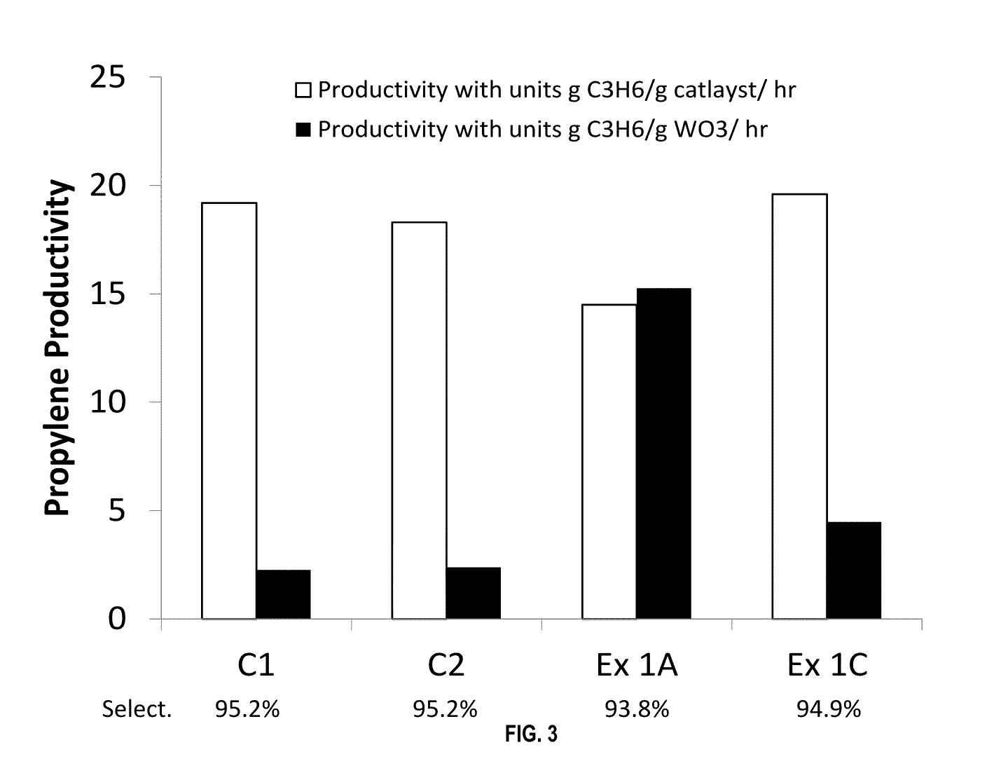

The propylene productivity and selectivity of the four samples is shown in FIG. 3 as both the productivity with respect to grams of total catalyst composite, and grams of tungsten alone. As seen from the figure, Example 1A exhibited 73% of the activity of the C1 catalyst for C₃H₆ production per gram of total catalyst composite, but did so with much less tungsten oxide. There are similar results with respect to C2. Thus, the propylene productivity per amount of tungsten oxide was almost an order of magnitude higher for Example 2. As seen from the figure, Example 1C exhibited equivalent activity of the C1 catalyst for C₃H₆ production per gram of total catalyst composite. This was achieved with ˜54% of the WO₃, showing a significantly greater WO₃ utilization. Moreover, Examples 1A and 1C exhibited comparable selectivity to C1 and C2.

As discussed above, it is thought that catalyst composites featuring the eggshell catalyst layer are able to increase the availability of the tungsten during the reaction. As a result, higher productivity for less catalyst can be achieved as shown in Examples 1A and 1C.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A catalyst composite comprising:
a support comprising at least about 90% by weight silica;
an eggshell layer on the support comprising about 0.25 to about 10 wt. % tungsten in the form of tungsten oxide or tungsten oxide hydrate, based on the total weight of the catalyst composite.

2. The catalyst composite of claim 1, wherein the tungsten oxide or tungsten oxide hydrate has an average crystal size of less than or equal to about 1 micron.

3. The catalyst composite of claim 2, wherein the tungsten oxide or tungsten oxide hydrate has an average crystal size of less than or equal to about 100 nm.

4. The catalyst composite of claim 1, wherein the catalyst composite exhibits an X-ray diffraction pattern comprising a peak at a two-theta value of about 16±0.5 degrees.

5. The catalyst composite of claim 1, wherein the catalyst composite exhibits an X-ray diffraction pattern comprising a peak at a two-theta value of about 26±0.5 degrees.

6. The catalyst composite of claim 1, exhibiting the X-ray diffraction pattern as shown in FIG. 1.

7. The catalyst composite of claim 1, wherein the catalyst composite comprises about 0.5 to about 7 wt. % tungsten in the form of tungsten oxide or tungsten oxide hydrate.

8. The catalyst composite of claim 1, wherein the eggshell layer has an average depth of 20 to 500 micrometers.

9. The catalyst composite of claim 8, wherein the eggshell layer has an average depth of 100 to 300 micrometers.

10. A method of making a catalyst composite, the method comprising:
providing a support comprising at least about 90% silica;
impregnating the support with water; and
impregnating the support with a solution comprising ammonium paratungstate and hydrochloric acid to provide a catalyst composite comprising silica and tungsten oxide hydrate.

11. The method of claim 10, further comprising drying the catalyst composite and calcining the catalyst composite at a temperature from about 150° C. to about 550° C. to provide a catalyst composite comprising silica and tungsten oxide.

12. The method of claim 10, wherein the catalyst composite comprises about 0.25 to about 10 wt. % tungsten oxide hydrate.

13. The method of claim 10, wherein the catalyst composite comprises about 0.5 to about 7 wt. % tungsten oxide hydrate.

14. The method of claim 10, wherein the support is impregnated by spraying the water on the support.

15. The method of claim 10, wherein the solution comprising ammonium paratungstate and hydrochloric acid is impregnated by spraying the solution on the support.

16. The method of claim 10, wherein the support is impregnated with about ¼ to about 1 pore volume of water and about ¼ to about 1 pore volume of the solution comprising ammonium paratungstate and hydrochloric acid.

17. The method of claim 10, wherein the molar ratio of the ammonium paratungstate to the hydrochloric acid in the solution is less than about 1:50.

18. A catalyst composite obtained by the method of claim 10.

19. A catalyst composite obtained by the method of claim 11.

20. A method of reacting olefins in a metathesis reaction, the method comprising contacting a stream comprising olefins with the catalyst composite of claim 1.

21. A method of reacting olefins in a metathesis reaction, the method comprising contacting a stream comprising olefins with the catalyst composite of claim 18.

22. A method of reacting olefins in a metathesis reaction, the method comprising contacting a stream comprising olefins with the catalyst composite of claim 19.
